(19) United States
(12) Patent Application Publication
(10) Pub. No.: US 2014/0188025 A1
(43) Pub. Date: Jul. 3, 2014

(54) Acupuncture Point Stimulating Compression Garment for Treatment of Musculoskeletal and Joint Pains

(71) Applicant: Mohammad Ahmed Aziz, Reseda, CA (US)

(72) Inventor: Mohammad Ahmed Aziz, Reseda, CA (US)

(21) Appl. No.: 13/731,068

(22) Filed: Dec. 30, 2012

(51) Int. Cl. A61F 5/05; A61F 5/01

(52) U.S. Cl. CPC A61F 5/05; A61F 5/0127; A61F 5/0123; A61F 5/013
USPC 602/21; 602/5; 602/27; 602/26; 602/20

(57) ABSTRACT

This invention is directed to an easy-to-use, convenient, and economical device and method for treating musculoskeletal and joint pain using acupuncture principles. An embodiment of the invention provides a compression support garment with an inner and outer surface, the garment being designed to be worn over a part of a person's body such that the inner surface covers at least an area of the part of the body experiencing pain, and wherein a plurality of beads are embedded into the inner surface of the garment at designated areas, the beads being capable of applying pressure to and stimulating acupuncture points on the connective tissue of the at least an area of a part of the body in contact with the beads according to traditional acupressure principles. Another embodiment discloses a method for treating pain that includes wearing a compression support garment with embedded acupuncture beads.

### BACKGROUND OF THE INVENTION

1. Field of the Invention

This invention generally relates to acupressure therapy, and in particular, to a device and method for treating musculoskeletal and joint pains by manipulating acupuncture points on the connective tissue of the body.

2. Description of the Related Art

Musculoskeletal and joint pains are common ailments. Musculoskeletal pain typically affects the muscles, ligaments, tendons, as well as the bones, while joint pain occurs in the connections (or joints) between the bones. The causes of musculoskeletal and joint pain are varied. Pain may occur in the muscles because of tissue damage from wear and tear of daily activities, repetitive movement, overuse, poor posture, prolonged immobilization, as well as from trauma such as jerking movements, auto accidents, fractures, falls, and sports-related injuries. For example, Medial Tibial Stress Syndrome (MTSS), commonly known as shin splints, is pain experienced in the lower leg on or around the tibia or shin bone. It is caused by continued stress or strain on the bone from athletic and/or military training. Specifically, the connective tissue around the shin bone is disturbed causing inflammation and reduced bone density. Along the same vein, joint pain can be caused by a number of different conditions such as osteoarthritis, rheumatoid arthritis, bursitis, gout, osteomyelitis, tendinitis, injuries such as fractures and sprains, as well as overuse and old age.

While short-term pain is for the most part bothersome and uncomfortable, chronic pain that lasts for weeks or months can debilitate a person's quality of life. Depending on the type and intensity, pain and inflammation in the muscles and joints can affect one's range of motion in and around the painful site, one's overall movement, as well as one's ability to carry out his or her daily activities with comfort and ease.

To relieve body and joint pains, people typically take over-the-counter, non-steroidal, anti-inflammatory drugs such as aspirin, ibuprofen, and acetaminophen. They also often apply topical creams such as Vicks®, Bengay® or other analgesic balms on the painful site to relieve joint and arthritic pain and other conditions. Some athletes use compression sleeves to relieve pain from shin splints and pain from sports-related injuries, but compression sleeves by themselves do not speed up recovery time, or provide significant pain relief.

With respect to treating MTSS, various studies have investigated the therapeutic effects of using ice application, aspirin, walking cast, gallium-arsenic laser, phenylbutazone, heel cord stretching, and legs braces. See Moen, M. H., et al., “The Treatment of Medial Tibial Stress Syndrome in Athletes: a Randomized Clinical Trial,” Sports Med. Arthrosc Rehabil Ther Technol., 4:12 (2012). These studies have shown, however, that the intervention group which received treatment did not recover significantly faster than the control group. Id. As such, there is currently no widely accepted treatment for MTSS and other types of musculoskeletal pain.

Typically, for chronic pain, physicians tend to prescribe muscle-relaxants such as Flexeril®, and pain killers such as Percocet® and Vicodin®. Other therapies for muscle and joint pain include injections with anesthetic or anti-inflammatory medicines in or around the painful and/or inflamed sites, physical therapy, occupational therapy, yoga and other stretching exercises, chiropractic treatment, therapeutic massage, acupressure massage, and acupuncture.

Acupuncture has been long known for its analgesic effects, which provide pain relief to patients with chronic joint and muscle pain. Even though acupuncture addresses disease and pain differently than Western Medicine, its use is becoming more prevalent in the West because of its therapeutic benefits. In the United States alone, some three million adults receive acupuncture treatments each year. See Vickers et al., “Acupuncture for Chronic Pain, Individual Patient Data Meta-analysis,” Arch. Intern. Med. (Sep. 10, 2012), www.archinternmed.com. Further, in Australia, eighty percent (80%) of general medical practitioners claim to have referred patients for acupuncture therapy to treat musculoskeletal and/or joint pain.

Acupuncture is performed by inserting extremely thin, solid needles in specific acupuncture points located along meridians in the skin. According to Traditional Chinese Medicine, acupuncture points lie on specific points on the body corresponding to specific meridians or channels. These meridians form a subtle energy system that circulates throughout the body. In other words, they are highways of “Chi” or energy pathways that run all over the body. According to Traditional Chinese Medicine, illness and pain are believed to occur when energy flow is disrupted within these meridians. Acupuncture is one tool that can be used to stimulate the meridians and restore the energy balance in the body, thereby alleviating pain and illness.

There are more than four hundred (400) acupuncture points and twenty (20) meridians connecting most of the points. In Asian countries, practitioners refer to acupuncture points by their names, and some points have several names. When acupuncture was adopted by Western society, however, a standard nomenclature was developed to describe acupuncture points. Under this nomenclature, an acupuncture point is identified by the meridian on which it is located and its number in the point sequence on that meridian. As such, points may often be described by the organs of the body that particular meridians pass through even though there is no direct effect on that specific organ, itself.

Although acupuncture points have no specific pattern, most of them lie on connective tissue. They are also known to stimulate connective tissue growth. For example, in a study performed on the mechanism of acupuncture needle grasp, the authors confirmed that, “needle rotation induced extensive fibroblast spreading and lamellipodia formation within 30 min, measurable as an increased in cell body cross sectional area.” See Langevin, et al., “Subcutaneous Tissue Fibroblast Cytoskeletal Remodeling Induced by Acupuncture: Evidence for a Mechanotransduction-based Mechanism,” J. of Cellular Physiol. (Mar. 1, 2006). Fibroblast is a component of the cytoskeleton that makes up connective tissue, and as Langevin et al. report, acupuncture has been shown to be effective in stimulating growth of connective tissue, thereby reducing inflammation caused by stress.

Modern science is still attempting to understand the mechanism by which acupuncture provides pain relief. For instance, much of the research at the University of California, Irvine Medical School's division of Complementary and Alternative Medicine is dedicated to studying the neuronal pathways involved in acupuncture-mediated pain relief.

Several theories involving different neuronal pathways and mechanisms of action have been proposed to describe how acupuncture provides pain relief. One theory is that stimulation of acupuncture points releases local nitric oxide, which is used by dilating blood vessels, causing increased blood flow, and in turn, increased nutrient exchange in the region. Another theory is that endorphins such as encephalin and dyanmin are released in the brain in response to varying frequency of stimulation of acupuncture points, and that these endorphins are used by the brain to lessen the perception of pain. Yet another theory posits that connective tissue is remodeled in response to acupuncture, allowing better support to joints and bones that are inflamed and in pain. See Langevin, et al., supra. While the exact mechanism of action is still unknown, the general consensus among the scientific community is that “acupuncture is a technique of peripheral sensory stimulation applied at acupuncture points . . . that can activate central nervous system pain pathways, release specific pain relieving substances as well as reduce muscle and sympathetic nervous system toxicity.” See Hinman et al., “Efficacy of Acupuncture for Chronic Knee Pain: Protocol for a Randomized Controlled Trial Using a Zelen Design,” BioMed Central Complementary and Alternative Medicine, 12:161 (2012).

Acupuncture has been recognized as a successful treatment for shin splints. For example, the Internet website Live Strong credits acupuncture as a successful treatment for shin splints based on several studies. According to Live Strong, in a 2002 study published in the Journal of Chinese Medicine, athletes with shin splints who underwent acupuncture reported significantly lower levels of pain than athletes who received traditional sports medicine treatments such as anti-inflammatory medication. Those who underwent acupuncture also found that pain was less of an interference with their playing sports. In another study published in the Medical Acupuncture Journal in 2001, stimulation of the stomach (45), small intestine (18) and gall bladder (44) acupuncture points resulted in complete recovery from shin splints within two days. See Acupuncture for Shin Splints at http://www.livestrong.com/article-534022-acup (Sep. 2, 2011).

Although there is significant support for the effectiveness of acupuncture for musculoskeletal and joint pains such as shin splits, most people typically avoid invasive procedures such as needle acupuncture. This may be due to perceptions of alternative medicine, as well as a fear of needles and what seems like an invasive procedure. Luckily, needle acupuncture is not the only way of stimulating acupuncture points along the meridians. Applying concentrated pressure on the acupuncture points through techniques such as acupressure massage can also bring about pain relief. While massaging acupressure points may not be as effective as stimulation with a needle, it can help ease pain from injury and can serve as a preventative measure. Routine massage often reduces pain and inflammation, and thus, is an effective way to manage pain and keep inflammation in check.

Regular massages at health spas can be expensive, however, so most people tend to view them as either luxuries or therapies reserved only for the most chronic pains. Auto-massagers that use electrodes are available in the market, but these devices do not stimulate acupuncture points. Rather, they massage the muscles at large. U.S. Pat. No. 8,137,379 for “Pressure Applying Device” describes a pliers-like device that can be used for applying pressure to specific meridians on a person's skin according to acupuncture principles. But, the pressure points are only stimulated while someone holds down the pliers-like device, making the device cumbersome and inconvenient to use.

Therefore, there is a need for a convenient, economical, and easy-to-use device and method for stimulating acupuncture points for pain relief without having to resort to a masseur or a needle acupuncturist.

### SUMMARY OF THE INVENTION

The present invention is thus directed to a device for treating musculoskeletal and joint pain that substantially obviates one or more of the problems due to limitations and disadvantages of the related art. As such, an object of the present invention is to provide a treatment for muscle and joint pain using acupuncture principles without having to seek the help of a masseur or acupuncture therapist. Another object of the present invention is to provide an easy-to-use, economical and convenient therapeutic remedy for musculoskeletal and joint pains.

Additional features and advantages of the invention will be set forth in the descriptions that follow and in part will be or shall become apparent from the description, or may be learned by practice of the invention. The objectives and other advantages of the invention will be realized and attained by the structure particularly pointed out in the written description and claims thereof as well as the accompanying drawings.

To achieve these and other advantages and in accordance with the purpose of the present invention, as embodied and broadly described, an embodiment of the present invention provides a device for treating musculoskeletal and joint pain comprising a compression support garment having an inner surface and an outer surface, wherein the garment is designed to be worn over a part of a person's body such that the inner surface of the garment covers at least an area of the part of the body experiencing pain, and wherein a plurality of beads are embedded into the inner surface of the garment at designated areas, the beads being capable of applying pressure to and stimulating acupuncture points on the connective tissue of the at least an area of the part of the body in contact with the beads according to traditional acupressure principles.

In another embodiment, the present invention provides a method for treating musculoskeletal or joint pain comprising wearing a compression support garment having an inner surface and an outer surface such that the inner surface of the garment covers at least an area of a part of the body experiencing pain, wherein the inner surface of the garment has a plurality of beads embedded into the surface at designated areas, the beads being capable of applying pressure to and stimulating acupuncture points on the connective tissue of at least an area of the part of the body in contact with the beads according to traditional acupuncture principles.

It is to be understood that both the foregoing general description and the following detailed description are merely exemplary, illustrative, and explanatory and as such, are intended to provide further explanation of the invention as claimed, and are not intended to limit the embodiments of the invention in any manner.

### BRIEF DESCRIPTION OF THE DRAWINGS

FIG. 1 is a cross-sectional view of a device according to an embodiment of this invention.

FIG. 2 depicts a right foot shown wearing a compression support ankle brace with embedded acupuncture beads according to an embodiment of this invention.

FIG. 3 is a front view of a right foot shown wearing a compression support ankle brace with embedded acupuncture beads according to an embodiment of this invention.

FIG. 4 is a front view of a right leg shown wearing a compression support knee sleeve with embedded acupuncture beads according to an embodiment of this invention.

FIG. 5 is a left side view of a right foot shown wearing a compression support ankle brace with embedded acupuncture beads according to an embodiment of this invention.

FIG. 6 is a back view of a right leg shown wearing a compression support knee sleeve with embedded acupuncture beads according to an embodiment of this invention.

FIG. 7 is a right side view of a right leg shown wearing a compression support knee sleeve with embedded acupuncture beads according to an embodiment of this invention.

FIG. 8 depicts the upper back of a person's body, shown wearing a compression support shoulder brace with embedded acupuncture beads according to an embodiment of this invention.

FIG. 9 illustrates an arm shown wearing a compression support arm sleeve with embedded acupuncture beads according to an embodiment of this invention.

FIG. 10 shows a top view of the arm shown wearing the same compression support sleeve as shown in FIG. 9.

FIG. 11 shows the inner arm with the same compression sleeve as shown in FIG. 9 and FIG. 10.

FIG. 12 illustrates a hand shown wearing a compression hand brace with embedded acupuncture beads according to an embodiment of this invention.

FIG. 13 is a top view of the hand shown wearing the same compression hand brace shown in FIG. 12.

FIG. 14 is a side view of the hand shown wearing the same compression support hand brace shown in FIG. 12 and FIG. 13.

FIG. 15 is a side view of an elbow shown wearing a compression support sleeve with embedded acupuncture beads according to an embodiment of this invention.

FIG. 16 illustrates the inner arm with the same compression support elbow sleeve shown in FIG. 15.

FIG. 17 depicts the outer arm and elbow with the same compression support sleeve shown in FIG. 15 and FIG. 16.

FIG. 18 depicts a front view of a right leg shown wearing a compression support shin sleeve with embedded acupuncture beads according to an embodiment of this invention.

FIG. 19 shows a right side view of a right leg shown wearing the same compression support shin sleeve shown in FIG. 18.

FIG. 20 shows a back view of a right leg shown wearing the same compression shin sleeve shown in FIG. 18 and FIG. 19.

### DETAILED DESCRIPTION OF THE PREFERRED EMBODIMENTS

The following detailed description is merely illustrative and exemplary in nature and is not intended to limit the embodiments of the subject matter or the application, or uses of such embodiments. As used in this disclosure, the words “exemplary” and “illustrative” mean “serving as an example, instance, or illustration.” Any implementation described as exemplary or illustrative is not meant to be necessarily construed as preferred or advantageous over other implementations. Further, there is no intention to be bound by any expressed or implied theory presented in the preceding background of the invention, brief summary, or the following detailed description.

As described earlier, an embodiment of the present invention is directed to a device for treating musculoskeletal and joint pain comprising a compression support garment having an inner surface and an outer surface, wherein the garment is designed to be worn over a part of a person's body such that the inner surface of the garment covers at least an area of the part of the body experiencing pain; and wherein a plurality of beads are embedded into the inner surface of the garment at designated areas, the beads being capable of applying pressure and stimulating acupuncture points on the connective tissue of at least an area of the part of the body in contact with the beads according to traditional acupressure techniques. In another embodiment, a method for treating musculoskeletal and joint pain generally comprises wearing a compression support garment with embedded beads capable of applying pressure and stimulating acupuncture points on connective tissue of the part of the body in contact with the beads.

In one embodiment of the present invention, the compression support garment is made of a stretchy, elasticized material. Suitable fabrics for the stretchy, uniformly elasticized material include nylon and polyester blend fabrics and neoprene. In a preferred embodiment, the outer surface of the garment may be composed of neoprene fabric while the inner surface that is in contact with the skin may be composed of a nylon or polyester blend material. The compression garment can be designed for different parts of the body experiencing pain, such as the ankle, the knee, the shin, the elbow, the lower arm, the wrist, the shoulder, the lower back, and so on. Thus, the device may comprise compression ankle braces, compression knee sleeves, compression wrist braces, compression shoulder compression braces, compression back braces, and so on.

The beads embedded in the compression garment can be made of a variety of suitable materials, including, among other things, solid round plastic beads, solid round wooden beads, solid round metal beads, and solid round gel beads. The beads are embedded at designated points within the inner surface of the compression such that they are capable of applying pressure to and stimulating specific acupuncture points when in contact with the wearer's skin. As such, the compression garments can be customized for different parts of the body and be designed to apply pressure on and stimulate specific acupuncture points on the connective tissue of the part of the body on, around, or over which the compression support garment is worn. The drawings exemplify how compression support garments with embedded acupressure beads according to an embodiment of this invention generally work.

A device for treating musculoskeletal and joint pain according to an embodiment of the present invention is shown in FIG. 1, which illustrates a cross sectional view of a compression support garment. As the drawing shows, the inner surface of the compression support garment generally comprises a nylon and/or polyester blend sheath. Round beads composed of wood, gel, metal, or plastic are embedded into the middle section, and sewn into the neoprene fabric that comprises the outer surface and provides elasticity and compression.

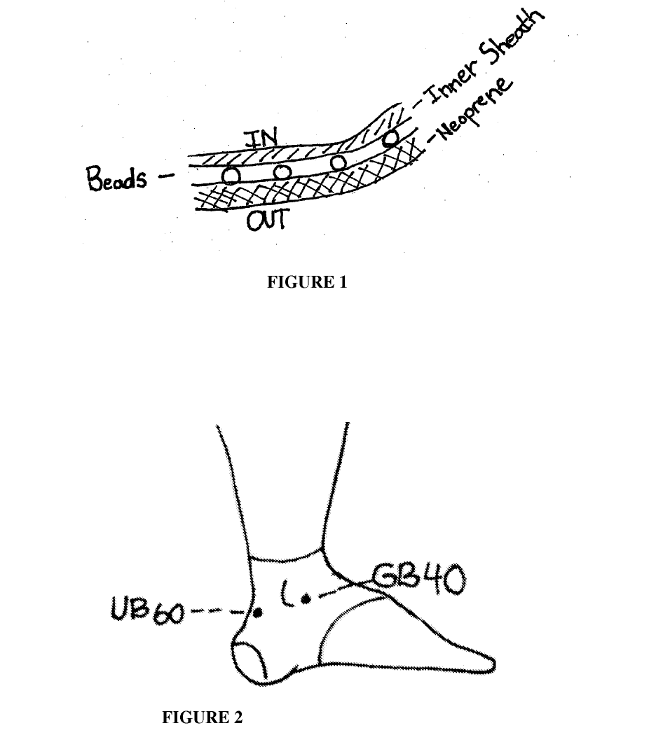

FIG. 2 illustrates a right side view of a compression support garment when worn over a right foot. In this embodiment, the device comprises compression ankle brace having an inner surface and an outer surface, wherein the inner surface of the compression brace covers the ankle, and wherein a plurality of beads are embedded into the inner surface of the compression brace at designated points. When the ankle brace is worn, the beads apply pressure on and stimulate acupressure points Urinary Bladder 60 (UB60) and Gall Bladder 40 (GB40) on the connective tissue of the ankle. The acupuncture points are named according to the meridians they lie upon. Under the nomenclature that has been adopted in Western countries, organs of the body are referenced in the names because the points pass through the respective organs without having any direct effect on the particular organ that is referenced.

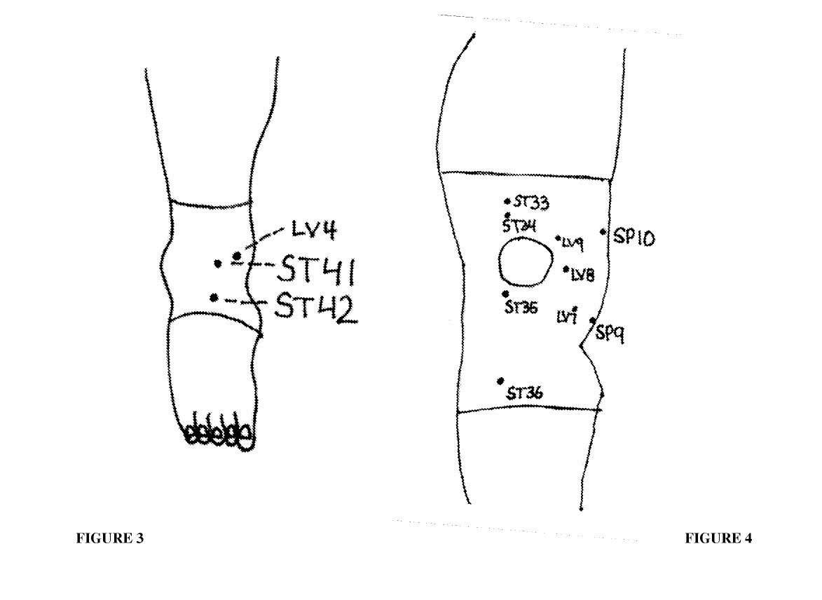

FIG. 3 is a front view of the same compression ankle brace with embedded acupres sure beads, showing the additional acupres sure points that are stimulated. As shown, when the ankle brace is worn, the plurality of beads embedded into the inner surface of the brace apply pressure on and stimulate acupressure points Liver 4 (LV4), Stomach 41 (ST41), and Stomach 42 (ST42) on the connective tissue of the ankle. FIG. 5 is a left side view of the right foot showing the same embodiment depicting a compression ankle brace with embedded acupressure beads, which apply pressure on and stimulate acupressure points Spleen 5 (SP5), Kidney 3 (KD3), Kidney 4 (KD4), Kidney 5 (KD5), and Kidney 6 (KD6) on the connective tissue of the ankle.

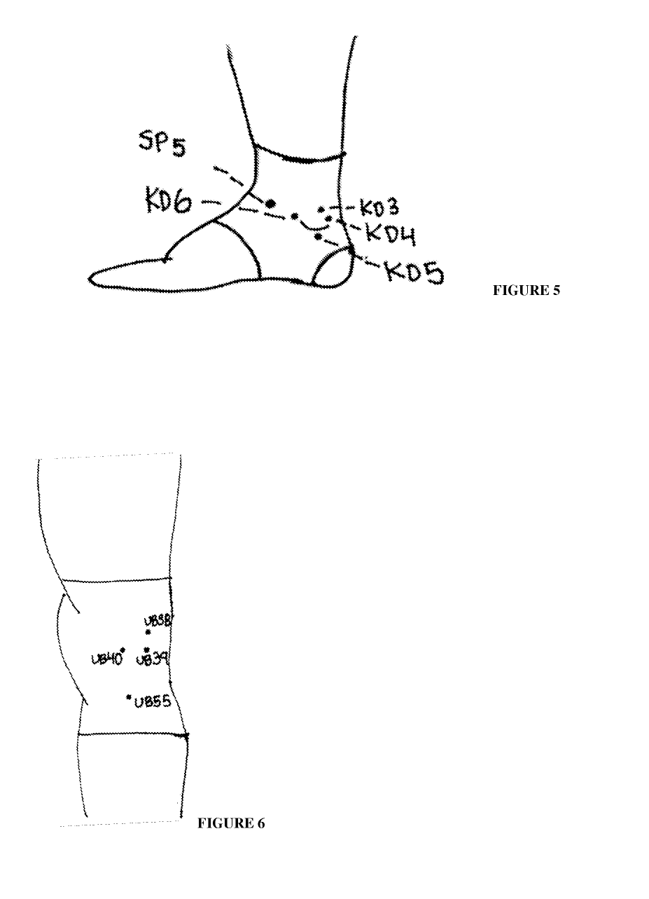

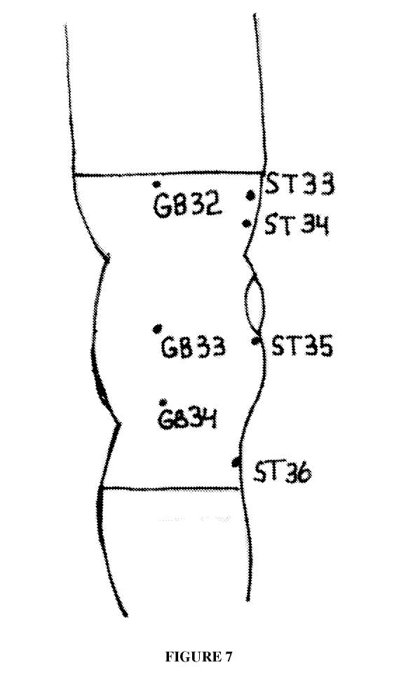

FIG. 4 depicts a front view of the right leg showing a compression support knee sleeve with embedded acupuncture beads according to an embodiment of this invention. As shown, when the compression support knee sleeve is worn over the knee, the beads embedded into the inner surface of the compression sleeve apply pressure on and stimulate acupuncture points Stomach 33 (ST33), Stomach 34 (ST34), Liver 9 (LV9), Spleen 10 (SP10), Liver 8 (LV8), Stomach 36 (ST36), Liver 7 (LV7), Spleen 9 (SP 9) and Stomach 36 (ST36) on the connective tissue of the knee. FIG. 6 illustrates the back of the right knee shown wearing the same compression support knee sleeve with embedded acupuncture beads, wherein the beads apply pressure on and stimulate acupuncture points Urinary Bladder 38 (UB38), Urinary Bladder 39 (UB39), Urinary Bladder 40 (UB40), and Urinary Bladder 55 (UB55) on the connective tissue of the back of the knee. FIG. 7 illustrates a right side of the right knee, shown wearing a compression support knee sleeve with embedded acupuncture beads. As shown in the drawing, when the compression support knee sleeve is worn, beads embedded into the inner surface of the sleeve apply pressure on and stimulate acupuncture points Gall Bladder 32 (GB32), Stomach 33 (ST33), Stomach 34 (ST34), Gall Bladder 33 (GB33), Stomach 35 (ST35), Gall Bladder 34 (GB34), and Stomach 36 (ST36) on the connective tissue of the knee.

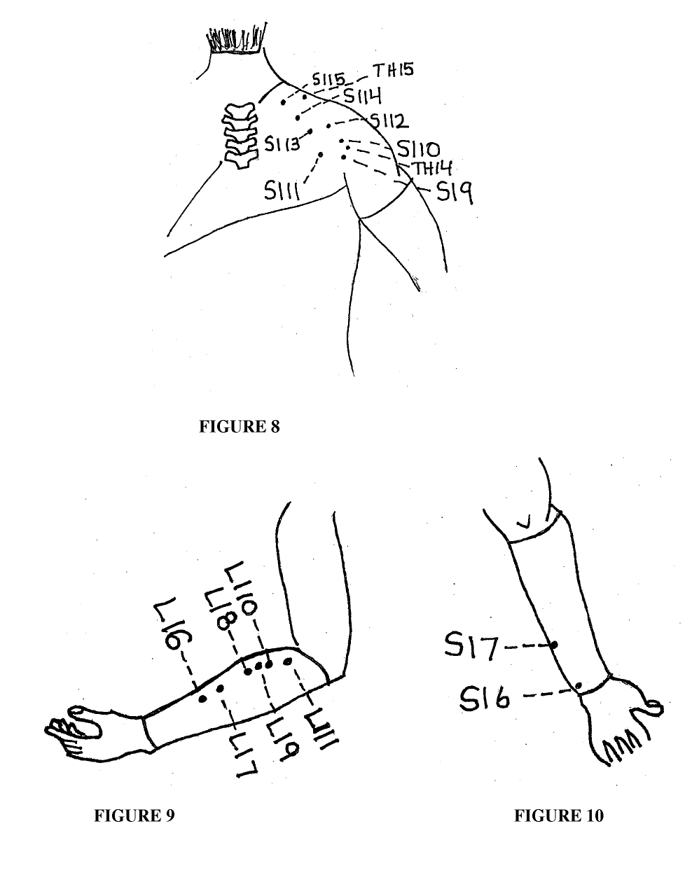

FIG. 8 illustrates a person's upper back, shown wearing a compression support shoulder brace with embedded acupuncture beads according to an embodiment of this invention. As shown, the acupuncture beads are embedded at designated points on the inner surface of the shoulder brace such that when the brace is worn, the beads apply pressure to and stimulate acupuncture points Small Intestine 9 (SI9), Small Intestine 10 (SI10), Small Intestine 11 (SI11), Small Intestine 12 (SI12), Small Intestine 13 (SI13), Small Intestine 14 (SI14), Small Intestine 15 (SI15), Triple Heater (TH14), and Triple Heater 15 (TH15) on the connective tissue on and around the shoulder blades.

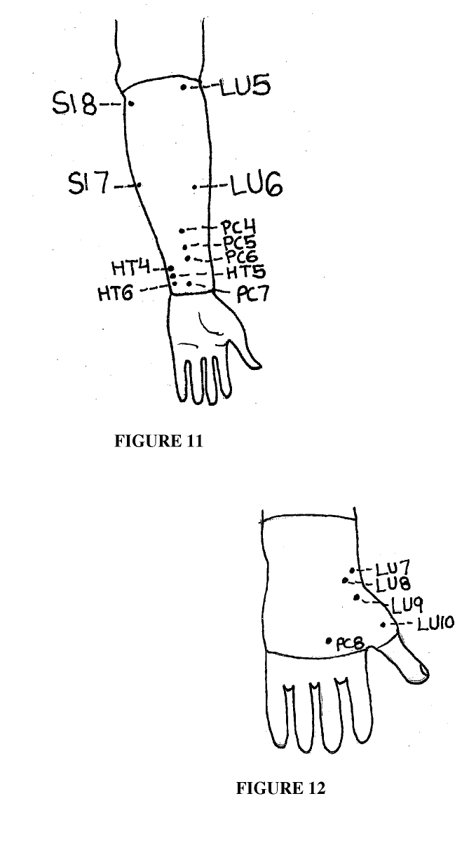

FIG. 9 depicts a side view of a left arm of a person, shown wearing a compression support sleeve that covers the arm from the elbow down to the wrist. As illustrated, beads embedded at designated points of inner surface of the sleeve apply pressure to and stimulate acupuncture points Large Intestine 10 (LI10), Large Intestine 11 (LI11), Large Intestine 18 (LI18), Large Intestine 19 (LI19), Large Intestine 16 (LI16), and Large Intestine 17 (LI17) on the connective tissue of the arm. FIG. 10 illustrates a top view of the arm, shown wearing the same compression sleeve with beads embedded at designated points which are designed to apply pressure to and stimulate acupuncture points Small Intestine 7 (SI7) and Small Intestine 6 (SI6). FIG. 11 depicts a bottom view of the left arm, showing the inner side of the left arm with the same compression support arm sleeve covering the arm from the elbow down to the wrist. As illustrated, beads embedded at designated points of the sleeve apply pressure to and stimulate acupuncture points Small Intestine 7 (SI7), Small Intestine 8 (SI8), Lung 5 (LU5), Lung 6 (LU6), Heart 4 (HT4), Heart 5 (HT5), and Heart 6 (HT6) in the connective tissue of the arm.

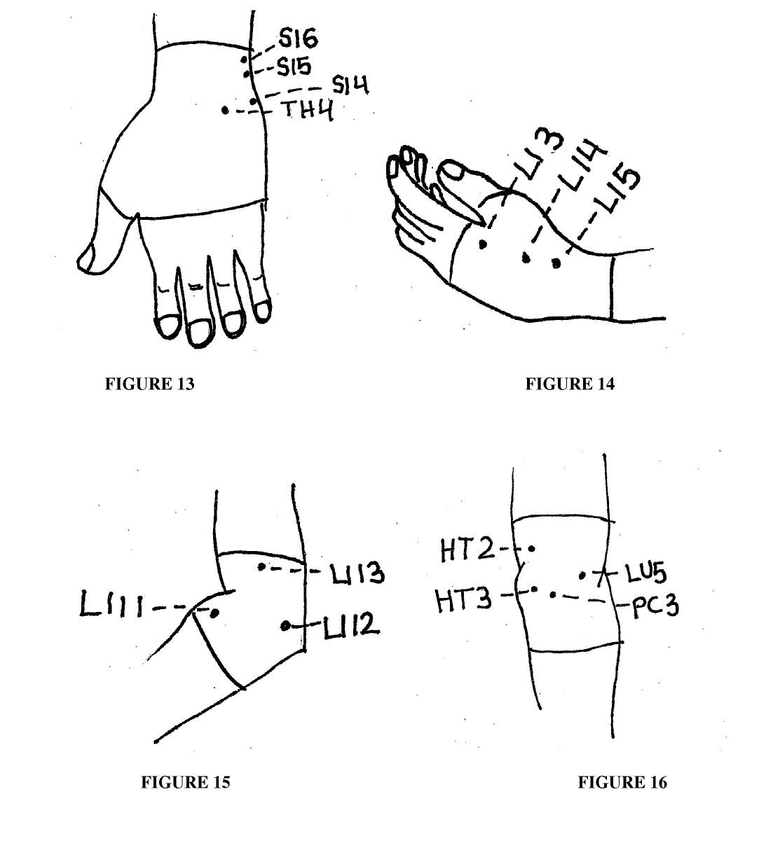

FIG. 12 illustrates a compression support hand brace with embedded acupuncture beads. In this embodiment, when the hand brace is worn, beads embedded into the surface of the brace apply pressure to and stimulate acupuncture points Pericardium 8 (PC8), Lung 7 (LU7), Lung 8 (LU8), Lung 9 (LU9), and Lung 10 (LU10) that lie on the meridian found on palm of the hand. FIG. 13 shows a top view of the same hand shown wearing the compression support hand brace with embedded acupuncture beads. As illustrated, the beads embedded at designated points apply pressure to and stimulate acupuncture points Small Intestine 14 (SI14), Small Intestine 15 (SI15), Small Intestine 6 (SI6), and Triple Heater 4 (TH4) that lie on the connective tissue of the hand. FIG. 14 shows a side view of the left hand wearing the same compression support hand brace, wherein beads embedded into the inner surface of the hand brace are designed to apply to and stimulate acupuncture points Large Intestine 3 (LI3), Large Intestine 4 (LI4), and Large Intestine 5 (LI5) according to an embodiment of this invention.

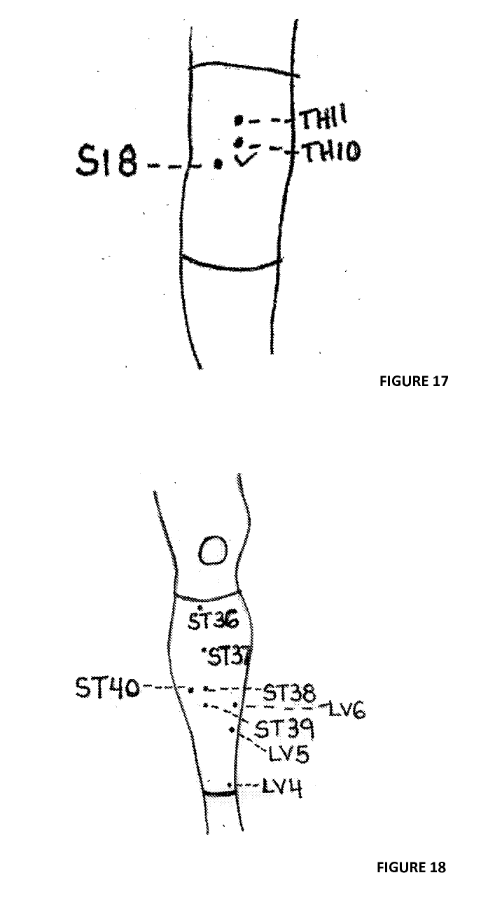

FIG. 15 illustrates a side view of the left elbow shown wearing a compression support elbow sleeve with embedded acupuncture beads, which are designed to apply pressure to and stimulate the acupuncture points Large Intestine 11 (LI11), Large Intestine 12 (LI12) and Large Intestine 13 (LI13) on the connective tissue of the arm. FIG. 16 shows the inner arm at the elbow with the same compression sleeve. As illustrated, embedded acupuncture beads in the sleeve apply pressure to and stimulate acupuncture points Heart 2 (HT2), Heart 3 (HT3), Lung 5 (LU5), and Pericardium 3 (PC3) according to an embodiment of this invention. FIG. 17 shows the outer arm and elbow with the same compression sleeve, illustrating that when the sleeve is worn, the embedded beads apply pressure to and stimulate acupuncture points Triple Heater 10 (TH10), Triple Heater 11 (TH11), and Small Intestine 8 (SI8).

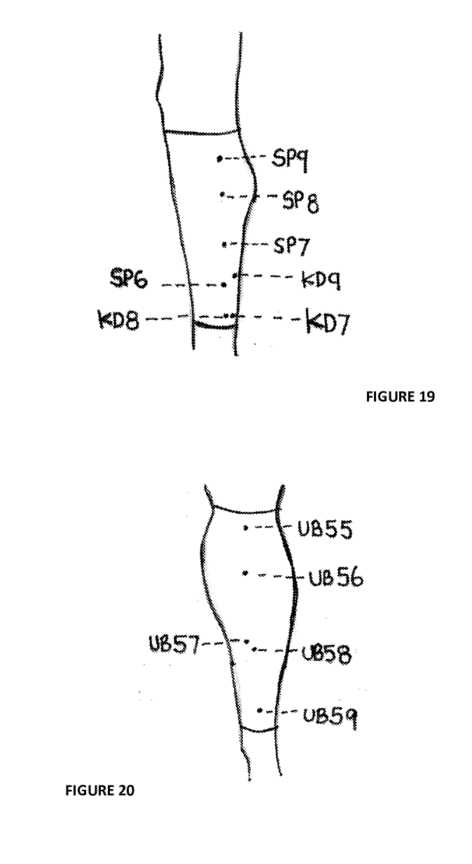

FIG. 18 is a front view of a compression shin sleeve with embedded acupuncture beads according to an embodiment of this invention. When worn, the beads embedded into the inner surface of the sleeve apply pressure to and stimulate acupuncture points Stomach 36 (ST36), Stomach 37 (ST37), Stomach 38 (ST38), Stomach 39 (ST39), Stomach 40 (ST40), Liver 4 (LV4), Liver 5 (LV5), and Liver 6 (LV6) on the connective tissue of the shin. FIG. 19 is a right side view of the right leg shown wearing the same compression shin sleeve with embedded acupuncture beads. As illustrated, when the shin sleeve is worn, the embedded beads also apply pressure to and stimulate acupuncture points Spleen 6 (SP6), Spleen 7 (SP7), Spleen 8 (SP8), Spleen 9 (SP9), Kidney 7 (KD7), Kidney 8 (KD8), and Kidney 9 (KD9). FIG. 20 is a back view of the leg shown wearing the same compression shin sleeve shown in FIG. 18 and FIG. 19. This figure illustrates that when the compression shin sleeve is worn, the embedded beads apply pressure to and stimulate acupuncture points Urinary Bladder 55 (UB55), Urinary Bladder 56 (UB56), Urinary Bladder 57 (UB57), Urinary Bladder 58 (UB58), and Urinary Bladder 59 (UB59) on the connective tissue in the back of the leg.

The following two examples illustrate how the devices and methods according to the embodiments of this invention are designed to perform:

### Example 1

Subject A was a forty-four (44) year-old female, who experienced pain predominantly in the calf muscles and in the feet, and notably, experienced vibrating pain in the heel and the middle of the foot. She did not take any pain medication for twenty-four (24) hours prior to treatment with the acupuncture stimulating compression sleeves. She was given a questionnaire to fill out regarding her pain levels and experience with the acupuncture point stimulating sleeves.

She described her baseline pain in the feet and calves (prior to treatment with the compression sleeves with embedded acupuncture beads) as noted in the following Table:

TABLE 1

| | Pain in Feet on a | Pain in Calves on a |
|---|---|---|
| Activity | Scale of 1-100 | Scale of 1-100 |
| Sitting down | 85/100 | 70/100 |
| Standing still | 90/100 | 70/100 |
| Walking | 90/100 | 70/100 |
| Exercise | 60/100 | 60/100 |
| (wall squats 30 reps; | | |
| calf raises 15 reps) | | |

Subject A's starting blood pressure was measured and recorded to be 100/72, and then, she put on compression support shin sleeves with embedded acupuncture beads as shown in FIGS. 19, 20, and 21. She wore the compression support shin sleeves with embedded acupuncture beads for fifty-five (55) minutes, and was asked to describe sensations in her feet and calves while wearing the sleeves.

She noted that immediately after putting on the shin sleeves, she felt a pinching sensation in her feet. After wearing the sleeve for about twenty-five (25) minutes, she said that the pinching sensation in the feet was almost gone, and at the end of the fifty-five (55) minutes, she said that the beads in the sleeves felt like they were digging in with less discomfort. At the end of the fifty-five minutes, the compression support shin sleeves were taken off, and Subject A's blood pressure was once again measured. It was recorded at 120/79.

Subject A was again asked to gauge her pain on a scale of 1-100 after wearing the compression sleeves with embedded acupuncture beads. She described the pain as noted in the following table:

TABLE 2

| | Pain in Feet on a | Pain in Calves on a |
|---|---|---|
| Activity | Scale of 1-100 | Scale of 1-100 |
| Sitting down | 20/100 | 30/100 |
| Standing still | 20/100 | 30/100 |
| Walking | 20/100 | 30/100 |
| Exercise | 20/100 | 30/100 |
| (wall squats 30 reps; | | |
| calf raises 15 reps) | | |

Subject A stated that she experienced a sensation of lightness or relaxation in the feet and calves after wearing the shin sleeves, and that she had better pain relief from wearing the compression support shin sleeves with embedded acupuncture beads than she had had from taking prescription and non-prescription pain killers.

### Example 2

Subject B was a fifty-six (56) year-old female who experienced pain in her lower calves and lower portion of the knees. She could not perform exercises such as squats and lunges as she had had knee replacement surgery. Subject B did not take any pain medication for twenty-four (24) hours prior to treatment. She was asked to fill out a questionnaire describing her pain and experience with the compression sleeve embedded with acupuncture beads. She described her baseline pain in the knees and calves (prior to treatment with the compression sleeves with embedded acupuncture beads) as noted in the following table:

TABLE 3

| | Pain in Knees on a | Pain in Calves on a |
|---|---|---|
| Activity | Scale of 1-100 | Scale of 1-100 |
| Sitting down | 55/100 | 60/100 |
| Standing still | 55/100 | 60/100 |
| Walking | 55/100 | 60/100 |

Subject B's starting blood pressure was measured and recorded to be 122/81. She then put on the compression support shin sleeves with embedded acupuncture beads as shown in FIGS. 19, 20, and 21, and noted her experiences in the questionnaire. She wore the compression support shin sleeves for a total of thirty (30) minutes.

She noted that immediately after putting on the shin sleeves, she felt a tingling sensation running down her legs. After wearing the sleeves for about ten (10) minutes, she felt like the beads were digging into her skin and causing slight discomfort. After walking for ten (10) minutes with the compression support shin sleeves shown in FIGS. 19, 20, and 21, Subject B reported that the discomfort from the beads had subsided. At the end of the thirty (30) minutes, the sleeves were taken off, and subject's blood pressure was once again measured. It was recorded at 122/76.

Subject B was asked to gauge her pain on a scale of 1-100 after wearing the knees for thirty (30) minutes. She described her pain as noted in the table below:

TABLE 4

| | Pain in Knees on a | Pain in Calves on a |
|---|---|---|
| Activity | Scale of 1-100 | Scale of 1-100 |
| Sitting down | 0/100 | 0/100 |
| Standing still | 0/100 | 0/100 |
| Walking | 0/100 | 0/100 |

Subject B stated that she experienced a tingling sensation in the knees and lower legs where the beads were in contact with the skin, and that she had pain relief and a sensation of lightness and relaxation in her leg muscles after wearing the compression support shin sleeves. She noted that the lightness in her legs felt different from the pain relief she had when she took prescription and non-prescription pain killers.

As the two examples above show, people experience pain relief through using the compression support garments with embedded acupuncture beads. In addition to the pain relief, subjects experienced a lightness or relaxation of muscles in the areas where the compression garment with the embedded beads were worn. As described, the length of treatment time with the compression support garments can be modified based on the type and intensity of the pain. Further, a person wearing the acupuncture point stimulating compression garments can sit still, lie down, walk, exercise, or carry out his or her routine activities while wearing the garments.

The above detailed description and examples present a description of the best mode contemplated of carrying out the present invention, and of the manner and process of making and using the invention in such full, clear, concise and exact terms so as to enable a person of ordinary skill in the art to make and use the invention. It will be apparent to those skilled in the art that various modifications, alternative constructions, and variations can be made in the device and the method of the present invention without departing from the spirit or scope of the invention. Thus, it is intended that the present invention cover modifications, alternative constructions, and variations that come within the spirit and scope of the invention as generally expressed by the following claims and their equivalents.

What is claimed is:

1. A device for treating musculoskeletal and joint pain comprising:
a compression support garment having an inner surface and an outer surface,
wherein the garment is designed to be worn over a part of a person's body such that the inner surface of the garment covers at least an area of the part of the body experiencing pain; and
wherein a plurality of beads are embedded into the inner surface of the garment at designated areas, the beads being capable of applying pressure to and stimulating acupuncture points on the connective tissue of the at least an area of the part of the body in contact with the beads according to traditional acupressure principles.

2. The device of claim 1 wherein the outer surface of the compression support garment is made of neoprene fabric.

3. The device of claim 1 wherein the inner surface of the compression support garment is made of a nylon or polyester blend fabric.

4. The device of claim 1 wherein the plurality of beads embedded into the inner surface of the garment comprise solid round beads made of plastic, wood, metal, or gel.

5. The device of claim 1 wherein the compression support garment comprises a stretchy elasticized brace designed to be worn around the ankle.

6. The device of claim 1 wherein the compression support garment comprises a stretchy elasticized sleeve designed to be worn around the shin.

7. The device of claim 1 wherein the compression support garment comprises a stretchy elasticized sleeve designed to be worn around the knee.

8. The device of claim 1 wherein the compression support garment comprises a stretchy elasticized sleeve designed to be worn around the elbow.

9. The device of claim 1 wherein the compression support garment comprises a stretchy elasticized brace designed to be worn around the wrist.

10. The device of claim 1 wherein the compression support garment comprises a stretchy elasticized brace designed to be worn around the shoulder.

11. A method of treating musculoskeletal or joint pain comprising:
wearing a compression support garment having an inner surface and an outer surface such that the inner surface of the garment covers at least an area of a part of the body experiencing pain,
wherein the inner surface of the garment has a plurality of beads embedded into the surface at designated areas, and
wherein the beads are capable of applying pressure and stimulating acupuncture points on the connective tissue of the at least an area of the part of the body in contact with the beads according to traditional acupuncture principles.

12. The method of claim 11 further comprising wearing the compression support garment around a shin.

13. The method of claim 11 further comprising wearing the compression support garment around a wrist.

14. The method of claim 11 further comprising wearing the compression support garment around a shoulder.

15. The method of claim 11 further comprising wearing the compression support garment around a knee.

16. The method of claim 11 further comprising wearing the compression support garment around an elbow.

17. The method of claim 11 further comprising wearing the compression support garment such that it covers a lower arm.
